(19) United States
(12) Patent Application Publication
(10) Pub. No.: US 2016/0033464 A1
(43) Pub. Date: Feb. 4, 2016

(54) METHODS FOR DETERMINING ACTIVE INGREDIENTS OF AN HERBAL MEDICINE, SOURCES OF AND CATALYTIC PATHWAYS FOR PRODUCTION THEREOF

(71) Applicant: Zongchao ZHANG, Norwood, NJ (US)

(72) Inventor: Zongchao ZHANG, Norwood, NJ (US)

(21) Appl. No.: 14/776,416

(22) PCT Filed: Mar. 7, 2014
(86) PCT No.: PCT/US2014/021885

§ 371 Date: Sep. 14, 2015

Related U.S. Application Data
(60) Provisional application No. 61/784,112, filed on Mar. 14, 2013.

(51) Int. Cl. G01N 33/15

(52) U.S. Cl. CPC G01N 33/15

(57) ABSTRACT

Disclosed are methods for determining active ingredients of herbal medicines including identifying organic sources and catalytic (mineral) pathways to produce such active ingredients.

### FIELD OF THE INVENTION

The presently disclosed and claimed inventive process(es), procedure(s), method(s), product(s), result(s) and/or concept(s) (collectively hereinafter referenced to as the “presently disclosed and claimed inventive concept(s)”) relates generally to herbal medicine prescriptions, and more particularly to methods for determining active ingredients of such herbal medicines including identifying organic sources and catalytic pathways to produce such active ingredients.

### DESCRIPTION OF THE RELATED ART

Herbs have been utilized for centuries for healing ailments ranging from arthritis to tuberculosis. Prescriptions of herbal medicines typically include a combination of herbs which together form an herbal medicine prescription, but such can include only one herb. Herbal medicine prescriptions are typically composed of lignocellulosic species, often in whole or in part, such as stems, roots, leaves, and seeds, of herbal grasses and shrubs, occasionally whole fruits or skins of plants, and in rare cases animal components such as armor or crust. Herbal medicine prescriptions have a long history of development in many parts of the world. Many prescriptions have been kept as family trade secrets. Such prescriptions remain more of an art than a science and their effectiveness has been mixed from case to case. Often, the effectiveness of a prescription for an ailment varies from patient to patient, and there is often poor understanding of the causes for the results. Many believe that the effectiveness of a prescription of herbal medicine depends on the origins where the herbs are grown and harvested. Herbal grasses and shrubs contain minerals absorbed from soil during growth. The mineral elements and the concentrations of the elements present in grasses and shrubs largely reflect the mineral content in the soil from which they grow. Minerals can be as high as 20% of the dry mass in certain grasses grown in mineral rich soils. The mineral element species cover the bulk of the elemental table, including some transition metals and some potentially harmful heavy metals. Elements such as Si, Al, Ca, Mg, Na, K, Ba, Sr, B, S, P, Cl, Cr, Mn, Fe, Co, Ni, Zn, Cu, Ti, Cd, Se, and Pb have all been reported.

A liquor, to be consumed by the patient, is formed from the herbs through a process called decoction. Herbal decoction is typically in boiling water and has traditionally been practiced in sandy pots by individual patients. The decoction process receives little control in the quality of water, pH, and the boiling temperature (as a result of varied mineral content). Decoction is usually performed in open air.

There has been tremendous interest in identifying and characterizing the active ingredients of herbal medicine prescriptions. The most prevalent methods appearing in patents and in literature are various extraction methods using extractive media under controlled conditions with the goal of extracting existing organics as medicinal candidates from the herbs. Such extracted organics have typically been further subjected to characterization and medicinal efficacy testing, such as animal or clinical testing. So far, such an approach has resulted in few successes.

Accordingly, there remains a need for an improved method and approach for determining active ingredients of herbal medicines, and to determine the organic source and catalytic pathways for production of such active ingredients to enable augmentation of the yield(s) of such.

### SUMMARY OF THE INVENTION

In accordance with an embodiment, a method is provided and comprises:
a) utilizing an herbal medicine comprising at least one herb comprising cellulosic material and minerals, wherein the cellulosic material comprises organic components, which include polysaccharides, lignin, peptides, alkaloids, and proteins;
b) analyzing each of the herbs of the herbal medicine to identify and quantify an analyte selected from the group consisting of: i) the minerals, ii) the elements: C, H, N, O, S, B, P, and halides, iii) the organic components, and iv) combinations thereof;
c) contacting a portion of each of the herbs of the herbal medicine with portions of a solvent under suitable conditions to remove any of the minerals removable by the solvent thereby forming mineral-reduced herbs, which can be without substantially removing the organic components;
d) analyzing each of the mineral-reduced herbs to identify and quantify non-removable minerals not removed in step c);
e) decocting a portion of each of the herbs in a portion of an aqueous solution comprising water to form individual herb liquors;
f) decocting a portion of each of the mineral-reduced herbs in a portion of the aqueous solution to form individual mineral-reduced herb liquors;
g) contacting a portion of the herbal medicine comprising each of the herbs with a portion of the solvent to remove any of the minerals removable by the solvent to form a mineral-reduced herbal medicine;
h) decocting a portion of the herbal medicine comprising each of the herbs in a portion of the aqueous solution to form an herbal medicine liquor;
i) decocting a portion of the mineral-reduced herbal medicine in a portion of the aqueous solution to form a mineral-reduced herbal medicine liquor;
j) performing medicinal efficacy testing, which can include animal and clinical testing, for each of the individual herb liquors, each of the individual mineral-reduced liquors, the herbal medicine liquor, and the mineral-reduced herbal medicine liquor; and
k) comparing the medicinal efficacies of the liquors tested in step j) to determine significant differences in medicinal efficacies and identifying liquors demonstrating significant differences in medicinal efficacy as effective liquors.

In accordance with another embodiment, a method of determining active ingredients of an herbal medicine is provided and comprises:
a) utilizing an herbal medicine comprising cellulosic material and minerals, wherein the cellulosic material comprises organic components, which include polysaccharides, lignin, peptides, alkaloids, and proteins;
b) decocting a portion of the herbal medicine in a portion of the aqueous solution to form a first liquor comprising organic compounds A;
c) analyzing the first liquor to identify the organic compounds A;
d) treating a portion of the herbal medicine to remove minerals therefrom to form a substantially mineral-free herbal medicine;
e) decocting a portion of the substantially mineral-free herbal medicine in a portion of the aqueous solution to form a second liquor comprising organic compounds B;
f) analyzing the second liquor to identify the organic compounds B;
g) comparing the organic compounds B with the organic compounds A and identifying organic compounds present in organic compounds A which are not present in the same amount as in organic compounds B resulting in a list of target organic compounds;
h) performing medicinal efficacy testing, which can include animal and clinical testing, for each of the target organic compounds; and
i) identifying each of the target organic compounds which demonstrate efficacy as an active ingredient of the herbal medicine.

### DETAILED DESCRIPTION OF THE INVENTION

In accordance with the presently disclosed and claimed inventive concept(s), herbal medicine prescriptions can comprise at least one herb comprising cellulosic material and minerals, wherein the cellulosic material can comprise organic components. The organic components can be selected from the group consisting of carbohydrates; polysaccharides; lignin; peptides; proteins; extractives such as steroids, fatty acids, fatty alcohols, alkylfrulates, terpenoids, phenolics, and alkaloids; and combinations thereof; and the organic components as a whole comprise elements C and H; and can further comprise at least one additional element selected from the group consisting of N, O, S, B, P, halides (such as F, Cl, Br, and I), and combinations thereof.

In the field of catalysis, it has been well established that some of such minerals present in various oxidation states as salts or as oxides or as hydroxides display catalytic functions that can enable certain transformations of the existing components in the herbs to take place under a wide range of conditions. The presence of alkali and alkaline earth metal ions in water can make water basic which can induce base catalysis, such as condensation reactions. The transition metals in oxide or in ionic form are also known to catalyze a wide range of transformations including, but not limited to, dehydration, hydrolytic cleavage of oxygen linkages, isomerization, anomerization, coupling, oxidation in the presence of air.

In accordance with an embodiment, the herbal medicine can be a prescription comprising a combination of individual herb species in prescribed weight for each in the prescription, or it can be a single herb.

An embodiment comprises the following steps:
a) utilizing the herbal medicine described above;
b) analyzing each of the herbs of the herbal medicine to identify and quantify an analyte selected from the group consisting of: i) the minerals, ii) the elements: C, H, N, O, S, B, P, and halides, iii) the organic components, and iv) combinations thereof;
c) contacting a portion of each of the herbs of the herbal medicine with portions of a solvent at suitable conditions to remove any of the minerals removable by the solvent thereby forming mineral-reduced herbs, which can be without substantially removing the organic components;
d) analyzing each of the mineral-reduced herbs to identify and quantify non-removable minerals not removed in step c);
e) decocting a portion of each of the herbs in a portion of an aqueous solution comprising water to form individual herb liquors;
f) decocting a portion of each of the mineral-reduced herbs in a portion of the aqueous solution to form individual mineral-reduced herb liquors;
g) contacting a portion of the herbal medicine comprising each of the herbs with a portion of the solvent to remove any of the minerals removable by the solvent to form a mineral-reduced herbal medicine;
h) decocting a portion of the herbal medicine comprising each of the herbs in a portion of the aqueous solution to form an herbal medicine liquor;
i) decocting a portion of the mineral-reduced herbal medicine in a portion of the aqueous solution to form a mineral-reduced herbal medicine liquor;
j) performing medicinal efficacy testing, which can include animal and clinical testing, for each of the individual herb liquors, each of the individual mineral-reduced liquors, the herbal medicine liquor, and said mineral-reduced herbal medicine liquor; and
k) comparing the medicinal efficacies of the liquors tested in step j) to determine significant differences in medicinal efficacies and identifying liquors demonstrating significant differences in medicinal efficacy as effective liquors.

Suitable conditions in step c) can include a temperature in the range of from about 0 to about 250° C., or about 50 to about 150° C., or about 80 to about 120° C.; and a pH in the range of from about 0.1 to about 14, or from about 3 to about 10, or from about 5 to about 9.

The solvent can be selected from the group consisting of water, an alcohol, an organic acid, an ester, a lactone, and combinations thereof, and the aqueous solution can further comprise a polar solvent, which can comprise an alcohol, an organic acid, an ester, a lactone, and combinations thereof.

The above described method of this embodiment can further comprise:
l) combining an additive mineral comprising at least one mineral selected from the group consisting of the minerals removed in step c) to a portion of the herbal medicine to form a mineral-enhanced herbal medicine;
m) decocting the mineral-enhanced herbal medicine in a portion of the aqueous solution to form a mineral-enhanced herbal medicine liquor;
n) performing medicinal efficacy testing for the mineral-enhanced herbal medicine liquor;
o) comparing the medicinal efficacy of the mineral-enhanced herbal medicine liquor with the medicinal efficacies of the liquors tested in step j) to determine significant differences in medicinal efficacies and, if the medicinal efficacy of the mineral-enhanced herbal medicine liquor is significantly enhanced over the medicinal efficacies of the liquors tested in step j), identifying the mineral-enhanced herbal medicine liquor as an effective liquor and identifying the additive mineral as an active mineral; and
p) repeating steps l)-o) at least once.

The above described method of this embodiment can further comprise:
q) combining an additive mineral comprising at least one mineral selected from the group consisting of the non-removable minerals to a portion of the herbal medicine to form a mineral-enhanced herbal medicine;
r) decocting the mineral-enhanced herbal medicine in a portion of the aqueous solution to form a mineral-enhanced herbal medicine liquor;
s) performing medicinal efficacy testing, which can include animal and clinical testing, for the mineral-enhanced herbal medicine liquor;
t) comparing the medicinal efficacy of the mineral-enhanced herbal medicine liquor with the medicinal efficacies of the liquors tested in step j) to determine significant differences in medicinal efficacies and, if the medicinal efficacy of the mineral-enhanced herbal medicine liquor is significantly enhanced over the medicinal efficacies of the liquors tested in step j), identifying the mineral-enhanced herbal medicine liquor as an effective liquor; and
u) repeating steps q)-t) at least once.

The above described method of this embodiment can further comprise
v) extracting and analyzing potential medicinal ingredients from each of the effective liquors to identify and quantify the potential medicinal ingredients;
w) performing medicinal efficacy testing, which can include animal and clinical testing, for each of the potential medicinal ingredients, and for combinations of the potential medicinal ingredients; and
x) comparing the results of the clinical testing in step w) for each of the potential medicinal ingredients and for each combination of the potential medicinal ingredients and identifying any of the potential medicinal ingredients or combinations of the potential medicinal ingredients which are effective as an effective medicinal ingredient or as an effective medicinal ingredient combination.

In accordance with another embodiment, a method of determining active ingredients of an herbal medicine comprises:
a) utilizing the herbal medicine as described above;
b) decocting a portion of the herbal medicine in a portion of the aqueous solution, as described above, to form a first liquor comprising organic compounds A;
c) analyzing the first liquor to identify the organic compounds A;
d) treating a portion of the herbal medicine to remove minerals therefrom to form a substantially mineral-free herbal medicine;
e) decocting a portion of the substantially mineral-free herbal medicine in a portion of the aqueous solution to form a second liquor comprising organic compounds B;
f) analyzing the second liquor to identify the organic compounds B;
g) comparing the organic compounds B with the organic compounds A and identifying organic compounds present in organic compounds A which are not present in the same amount as in organic compounds B resulting in a list of target organic compounds;
h) performing medicinal efficacy testing, which can include animal and clinical testing, for each of the target organic compounds; and
i) identifying each of the target organic compounds which demonstrate efficacy as an active ingredient of the herbal medicine.

The above described method of this embodiment can further comprise:
j) analyzing a portion of the herbal medicine to identify and quantify the minerals resulting in a list of identified minerals;
k) adding one of the identified minerals or a combination of the identified minerals to a portion of the mineral-free herbal medicine to form a mineral spiked herbal medicine;
l) decocting the mineral spiked herbal medicine in a portion of the aqueous solution to form a mineral spiked liquor comprising mineral spiked organic compounds;
m) analyzing the mineral spiked liquor to identify the mineral spiked organic compounds;
n) comparing the mineral spiked organic compounds with the active ingredients to identify any matches resulting in identification of one of the identified minerals or combination of the identified minerals present in the herbal medicine as an active mineral or as an active mineral combination active in the production of at least one of the active ingredients;
o) repeating steps k)-n) at least once.

The decocting steps described above can be performed at any suitable conditions to accomplish decocting. Such suitable conditions for decocting can include a temperature in the range of from about 0 to about 250° C., or about 50 to about 150° C., or about 80 to about 120° C.; and a pH in the range of from about 0.1 to about 14, or from about 3 to about 10, or from about 5 to about 9. Also, such suitable conditions for decocting can include temperature and pressure conditions sufficient to boil the aqueous solution. The pressure can exceed ambient. The conventional herbal medicine decoction is typically carried out at local ambient pressure to the boiling point of the aqueous solution. By performing the decocting steps in an apparatus at elevated or high pressure the decocting temperature can be controlled to allow high temperature decoction. The decocting temperature can be optimized in such apparatus to maximize the yield of active medicinal compounds.

The liquors formed above from decoction of the herbal medicines can be separated from the residues of the herbal medicines by any separation method known to separate a liquid from a solid, and more specifically, by a method selected from the group consisting of filtration, decanting, pressurized filtration, and combinations thereof.

In conventional decocting of herbal medicines, minerals can be left in the medicinal liquor. These minerals can be optionally removed by various methods from the liquors. The minerals or mineral ions can be removed by a method selected from the group consisting of ion exchange with protonic polymeric resins, precipitation with the aid of precipitating reagents (such as for heavy metals), chelation using chelating reagents, and combinations thereof.

The precipitating reagents can include, but are not limited to, carbon dioxide, alkali carbonates, etc.

The above described method of this embodiment can further comprise:
p) analyzing a portion of the herbal medicine to identify and quantify the organic components;
q) isolating each of the organic components from a portion of the herbal medicine;
r) exposing one of the organic components or a combination of the organic components with one of the active minerals or one of the active mineral combinations under conversion conditions to form a product stream comprising product organic compounds;
s) analyzing the product stream to identify and quantify the product organic compounds;
t) comparing the product organic compounds with the active ingredients to identify any matches resulting in identification of one of the organic components or combination of the organic components present in the herbal medicine as a source organic component or source organic component combination convertible to at least one of the active ingredients;
u) repeating steps r)-t) at least once.

Applicant believes that certain mineral elements in an herbal medicine prescription, containing at least one herb and usually a mix of multiple herbs, catalyze the transformation of natural components present in the herbs during the decoction process to produce new molecules that are medicinally active for the treatment of certain diseases. During the process of herb decoction in water, Applicant believes that the natural components of herbs undergo a catalytic transformation process to produce new molecules, some of which are active medicinal ingredients, due to the presence of certain minerals embedded in the mass of the herbs. The catalyzed transformation could be the re-arrangement of certain bonds in a natural component of a single herb or the elimination of a certain functional group(s) in a natural component of a single herb, or the condensation of two or multiple components derived from different herb species.

The methods described above are intended to provide practicable examples of general methods. The presently disclosed and claimed inventive concept(s) should not be limited by the above listed steps. Adding and removing minerals before or during decoction of herbs are examples of verifying the catalytic functions of the minerals. Contacting with an aqueous solution is just an optional method to remove certain minerals. There is no fixed order in the procedure to achieve this goal. Improved medicinal efficacy achieved through this invention by verifying catalytic effect of certain minerals on the conversions of the original natural herb components and further identification and determination of the structure of the potential new drug molecules using various typical physical methods such as NMR, FTIR, Raman, GC-MS, LC-MS, GPC, etc. are all associated with this invention.

Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

Further, unless expressly stated otherwise, the term “about” as used herein is intended to include and take into account variations due to manufacturing tolerances and/or variabilities in process control.

Changes may be made in the construction and the operation of the various components, elements and assemblies described herein, and changes may be made in the steps or sequence of steps of the methods described herein without departing from the spirit and the scope of the invention as defined in the following claims.

What is claimed is:

1. A method comprising:
a) utilizing an herbal medicine comprising at least one herb comprising cellulosic material and minerals, wherein said cellulosic material comprises organic components;
b) analyzing each of said herbs of said herbal medicine to identify and quantify an analyte selected from the group consisting of: i) said minerals, ii) the elements: C, H, N, O, S, B, P, and halides, iii) said organic components, and iv) combinations thereof;
c) contacting a portion of each of said herbs of said herbal medicine with portions of a solvent to remove any of said minerals removable by said solvent thereby forming mineral-reduced herbs;
d) analyzing each of said mineral-reduced herbs to identify and quantify non-removable minerals not removed in step c);
e) decocting a portion of each of said herbs in a portion of an aqueous solution comprising water to form individual herb liquors;
f) decocting a portion of each of said mineral-reduced herbs in a portion of said aqueous solution to form individual mineral-reduced herb liquors;
g) contacting a portion of said herbal medicine comprising each of said herbs with a portion of said solvent to remove any of said minerals removable by said solvent to form a mineral-reduced herbal medicine;
h) decocting a portion of said herbal medicine comprising each of said herbs in a portion of said aqueous solution to form an herbal medicine liquor;
i) decocting a portion of said mineral-reduced herbal medicine in a portion of said aqueous solution to form a mineral-reduced herbal medicine liquor;
j) performing medicinal efficacy testing for each of said individual herb liquors, each of said individual mineral-reduced liquors, said herbal medicine liquor, and said mineral-reduced herbal medicine liquor; and
k) comparing the medicinal efficacies of the liquors tested in step j) to determine significant differences in medicinal efficacies and identifying liquors demonstrating significant differences in medicinal efficacy as effective liquors.

2. The method of claim 1 wherein said organic components are selected from the group consisting of polysaccharides, carbohydrates, lignin, peptides, extractives, proteins, and combinations thereof; and wherein said organic components as a whole comprise elements C and H.

3. The method of claim 2 wherein said organic components further comprise at least one additional element selected from the group consisting of N, O, S, B, P, halides, and combinations thereof.

4. The method of claim 1 wherein said solvent is selected from the group consisting of water, an alcohol, an organic acid, an ester, a lactone, and combinations thereof.

5. The method of claim 1 wherein said aqueous solution further comprises a polar solvent.

6. The method of claim 5 wherein said polar solvent comprises an alcohol, an organic acid, an ester, a lactone, and combinations thereof.

7. The method of claim 1 further comprising:
l) combining an additive mineral comprising at least one mineral selected from the group consisting of said minerals removed in step c) to a portion of said herbal medicine to form a mineral-enhanced herbal medicine;
m) decocting said mineral-enhanced herbal medicine in a portion of said aqueous solution to form a mineral-enhanced herbal medicine liquor;
n) performing medicinal efficacy testing for said mineral-enhanced herbal medicine liquor;
o) comparing the medicinal efficacy of said mineral-enhanced herbal medicine liquor with the medicinal efficacies of the liquors tested in step j) to determine significant differences in medicinal efficacies and, if the medicinal efficacy of said mineral-enhanced herbal medicine liquor is significantly enhanced over the medicinal efficacies of the liquors tested in step j), identifying said mineral-enhanced herbal medicine liquor as an effective liquor and identifying said additive mineral as an active mineral; and
p) repeating steps l)-o) at least once.

8. The method of claim 7 wherein steps e), f), h), i) and m) are each performed at suitable conditions for decocting including a temperature in the range of from about 0 to about 250° C., and a pH in the range of from about 0.1 to about 14.

9. The method of claim 7 wherein steps e), f), h), i) and m) are each performed at temperature and pressure conditions sufficient to boil said aqueous solution.

10. The method of claim 9 wherein the pressure exceeds ambient.

11. The method of claim 7 wherein each of the liquors formed in steps e), f), h), i) and m) are separated from the herbal medicines after decocting by a method selected from the group consisting of filtration, decanting, pressurized filtration, and combinations thereof.

12. The method of claim 7 wherein minerals are removed from the liquors formed in steps e), f), h), i) and m) by a method selected from the group consisting of ion exchange with protonic polymeric resins, precipitation with the aid of precipitating reagents, chelation with chelating reagents, and combinations thereof.

13. The method of claim 7 further comprising:
q) combining an additive mineral comprising at least one mineral selected from the group consisting of said non-removable minerals to a portion of said herbal medicine to form a mineral-enhanced herbal medicine;
r) decocting said mineral-enhanced herbal medicine in a portion of said aqueous solution to form a mineral-enhanced herbal medicine liquor;
s) performing medicinal efficacy testing for said mineral-enhanced herbal medicine liquor;
t) comparing the medicinal efficacy of said mineral-enhanced herbal medicine liquor with the medicinal efficacies of the liquors tested in step j) to determine significant differences in medicinal efficacies and, if the medicinal efficacy of said mineral-enhanced herbal medicine liquor is significantly enhanced over the medicinal efficacies of the liquors tested in step j), identifying said mineral-enhanced herbal medicine liquor as an effective liquor; and
u) repeating steps q)-t) at least once.

14. The method of claim 13 further comprising:
v) extracting and analyzing potential medicinal ingredients from each of said effective liquors to identify and quantify said potential medicinal ingredients;
w) performing medicinal efficacy testing for each of said potential medicinal ingredients, and for combinations of said potential medicinal ingredients; and
x) comparing the results of the medicinal efficacy testing in step w) for each of said potential medicinal ingredients and for each combination of said potential medicinal ingredients and identifying any of said potential medicinal ingredients or combinations of said potential medicinal ingredients which are effective in treating a patient as an effective medicinal ingredient or as an effective medicinal ingredient combination.

15. A method of determining active ingredients of an herbal medicine comprising:
a) utilizing an herbal medicine comprising cellulosic material and minerals, wherein said cellulosic material comprises organic components;
b) decocting a portion of said herbal medicine in a portion of an aqueous solution comprising water to form a first liquor comprising organic compounds A;
c) analyzing said first liquor to identify said organic compounds A;
d) treating a portion of said herbal medicine to remove minerals therefrom to form a substantially mineral-free herbal medicine;
e) decocting a portion of said substantially mineral-free herbal medicine in a portion of said aqueous solution to form a second liquor comprising organic compounds B;
f) analyzing said second liquor to identify said organic compounds B;
g) comparing said organic compounds B with said organic compounds A and identifying organic compounds present in organic compounds A which are not present in the same amount as in organic compounds B resulting in a list of target organic compounds;
h) performing medicinal efficacy testing for each of said target organic compounds; and
i) identifying each of said target organic compounds which demonstrate efficacy in treating a patient as an active ingredient of said herbal medicine.

16. The method of claim 15 wherein said organic components are selected from the group consisting of polysaccharides, carbohydrates, lignin, peptides, extractives, proteins, and combinations thereof; and wherein said organic components as a whole comprise elements C and H.

17. The method of claim 16 wherein said organic components further comprise at least one additional element selected from the group consisting of N, O, S, B, P, halides, and combinations thereof.

18. The method of claim 15 wherein said aqueous solution further comprises a polar solvent.

19. The method of claim 18 wherein said polar solvent comprises an alcohol, an organic acid, an ester, a lactone, and combinations thereof.

20. The method of claim 15 further comprising:
j) analyzing a portion of said herbal medicine to identify and quantify said minerals resulting in a list of identified minerals;
k) adding one of said identified minerals or a combination of said identified minerals to a portion of said mineral-free herbal medicine to form a mineral spiked herbal medicine;
l) decocting said mineral spiked herbal medicine in a portion of said aqueous solution to form a mineral spiked liquor comprising mineral spiked organic compounds;
m) analyzing said mineral spiked liquor to identify said mineral spiked organic compounds;
n) comparing said mineral spiked organic compounds with said active ingredients to identify any matches resulting in identification of one of said identified minerals or combination of said identified minerals present in said herbal medicine as an active mineral or as an active mineral combination active in the production of at least one of said active ingredients; and
o) repeating steps k)-n) at least once.

21. The method of claim 20 wherein steps b), e) and l) are each performed at suitable conditions for decocting including a temperature in the range of from about 0 to about 250° C., and a pH in the range of from about 0.1 to about 14.

22. The method of claim 20 wherein steps b), e), and l) are each performed at temperature and pressure conditions sufficient to boil said aqueous solution.

23. The method of claim 22 wherein the pressure exceeds ambient.

24. The method of claim 20 wherein each of the liquors formed in steps b), e) and l) are separated from the herbal medicines after decocting by a method selected from the group consisting of filtration, decanting, pressurized filtration, and combinations thereof.

25. The method of claim 20 wherein minerals are removed from the liquors formed in steps b), e) and l) by a method selected from the group consisting of ion exchange with protonic polymeric resins, precipitation with the aid of precipitating reagents, chelation with chelating reagents, and combinations thereof.

26. The method of claim 20 further comprising:
p) analyzing a portion of said herbal medicine to identify and quantify said organic components;
q) isolating each of said organic components from a portion of said herbal medicine;
r) exposing one of said organic components or a combination of said organic components with one of said active minerals or one of said active mineral combinations under conversion conditions to form a product stream comprising product organic compounds;
s) analyzing said product stream to identify and quantify said product organic compounds;
t) comparing said product organic compounds with said active ingredients to identify any matches resulting in identification of one of said organic components or combination of said organic components present in said herbal medicine as a source organic component or source organic component combination convertible to at least one of said active ingredients;
u) repeating steps r)-t) at least once.
